SEQUENCE LISTING (4 sequences)

SEQ ID NO: 1 - DNA, 5386 bp, Artificial sequence
  Multicistronic plasmid vector
attattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattatccgctagctcccggccgccatggcggccgcgggaattcgattgatcgatcgagagaaatgttctggcacctgcacttgcactggggacagcctattttgctagtttgttttgtttcgttttgttttgatggagagcgtatgttgctagcaccggtcatggctgcgccccgacacccgccaacacccggtgacgcgccctgacgggcttgtctgctcccggcatccgcttacagacaagctgtgaccgtctccgggagctgcatgtgtcagaggttttcaccgtcatcaccgaaacgcgcgaggcagccggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgcgtacggaaattcgagctcggtacccggggatcctttaaatgtggcaagcttatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcggaattggccgccctagatgctcgaggatcctctagagtcgacctgcagaaaagacccatggaaaggaacagtctgttagtctgtcagctattatgtctggtggcgcgcgcggcagcaacgagtactgctcagactacactgccctccaccgttaacagcaccgcaacgggagttacctctgactcttatcagaatacaacaactcagctgcctgcatcttcttctgccgctgccttaagtcttccatctgcgtcagccgtgcgagcccaatctccacgctcattttcagacacataccctaccgccacggccttgtgcggcacactggtggtggtgggcattgtgctgtgcctaagtctggcctccactgttaggagcaaggagctgccgagcgaccatgagccgctggaggcatgggaccagggctcggatgtggaagctccgccgctaccggagaagagcccatgtccggaacacgtacccgagattcgcgtggagatcccacgctatgtttaataaaaactgcgggcacgggggacggcgttgttgtatatgtgaatttgtaaataataaatgggaccccatcctgtaaaaatacagagtccgtgtcagtctctgaaggacagagtattggcatatagccaatagagatagttgtggcaaagagccatgttatggattagtaatggaaagtatcgtcaccaataggggagtggtcaataatggtcaataacccacacctataggctaagctataccatcacctatagcataaggaagcgggggtgtataggccccaagccaaaaacagtatagcatgcataagagccaaaggggtgtgcctatagactctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacatggcggtaatgttggacatgagccaatataaatgtacatattatgatatggatacaacgtatgcaatggccactgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaatt

SEQ ID NO: 2 - DNA, 4457 bp, Artificial sequence
  Multicistronic plasmid vector
attattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgttgacagcttatcatcgcagatcctgagcttgtatggtgcactctcagtacaatctgctctgctgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagataattgtatttaagtgcctagctcgatacaataaacgccatttgaccattcaccacattggtgtgcacctccaagctgggtagctagctcgatcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaattcctcgacggatgatatcttcgaagcctccaggcccccccctcccgggagagccatagtggtctgcggaaccggtgagtacaccggaattaccggaaagactgggtcctttcttggataaacccactctatgtccggtcatttgggcgtgcccccgcaagactgctagccgagtagcgttgggttgcgaaaggccttgtggtactgcctgatagggtgcttgcgagtgccccgggaggtctcttagaccgtgcaagcggccgcgccattcaggctggatccagatctacttctggctaataaaagatcagagctctagagatctgtgtgttggttttttgtgtggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagatgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaatt

SEQ ID NO: 3 - DNA, 7086 bp, Artificial sequence
  Multicistronic plasmid vector
attattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcatctatgatttctctcagtacttcctcacatgttggaggcattttctctccctctgcacttaatagcctatcttgctttaatttcttcccactcttggaggctaggtttggtttggtgggctgatgagggagggagagaccgctccaagtactttagcgggtgggattgaaggcggagccctcctgagctatgagtgtcctatgagtggggctggaactaagaaccaggggcgtggacagggtgtgtcacagagaaggggatgtgcctgcttctttctggcccaggaggaaccgggtcaattcttcagcacctgggtaccacacaaaaaaccaacacacagatctctagagctctgatcttttattagccagaagtagatctggatccagcctgaatggcgcggcctcgaatcaggcctaaaattaattaaaatgatgagacagcacaacaaccagcacgttgcccaggagctgtaggaaagagaagaaggcatgaacatggttagcagaggggcccggtttggactcagagtattttatcctcatctcaaacagtgtatatcattgtaaccataaagagaaaggcaggatgatgaccagggtgtagttgtttctaccaataagaatatttccacgccagccagaatttatatgcagaaatattctaccttatcatttaattataacaattgttctctaaaactgtgctgaagtacaatataatataccctgattgccttgaaaaaaaagtgattagagaaagtacttacaatctgacaaataaacaaaagtgaatttaaaaattcgttacaaatgcaagctaaagtttaacgaaaaagttacagaaaatgaaaagaaaataagaggagacaatggttgtcaacagagtagaaagtgaaagaaacaaaattatcatgagggtccatggtgatacaagggacatcttcccattctaaacaacaccctgaaaactttgccccctccatataacatgaattttacaatagcgaaaaagaaagaacaatcaagggtccccaaactcaccctgaagttctcaggatcggtcgatcgagctagctacccagcttggaggtgcacaccaatgtggtgaatggtcaaatggcgtttattgtatcgagctaggcacttaaatacaatatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacatttccccctccctatgcaaaagcgaaactactatatcctgaggggactcctaaccgcgtacaaccgaagccccgcttttcgcctaaacacaccctagtcccctcagatacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccatacggatctgcgatgataagctgtcaacatagcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattatccgctagctcccggccgccatggcggccggccgcgggaattcgattgatcgatcgagagaaatgttctggcacctgcacttgcactggggacagcctattttgctagtttgttttgtttcgttttgttttgatggagagcgtatgttgctagcaccggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagccggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgcgtacggaaattcgagctcggtacccggggatcctttaaatgtggcaagcttatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcggaattggccgccctagatgctcgaggatcctctagagtcgacctgcagaaaagacccatggaaaggaacagtctgttagtctgtcagctattatgtctggtggcgcgcgcggcagcaacgagtactgctcagactacactgccctccaccgttaacagcaccgcaacgggagttacctctgactcttatcagaatacaacaactcagctgcctgcatcttcttctgccgctgccttaagtcttccatctgcgtcagccgtgcgagcccaatctccacgctcattttcagacacataccctaccgccacggccttgtgcggcacactggtggtggtgggcattgtgctgtgcctaagtctggcctccactgttaggagcaaggagctgccgagcgaccatgagccgctggaggcatgggaccagggctcggatgtggaagctccgccgctaccggagaagagcccatgtccggaacacgtacccgagattcgcgtggagatcccacgctatgtttaataaaaactgcgggcacgggggacggcgttgttgtatatgtgaatttgtaaataataaatgggaccccatcctgtaaaaatacagagtccgtgtcagtctctgaaggacagagtattggcatatagccaatagagatagttgtggcaaagagccatgttatggattagtaatggaaagtatcgtcaccaataggggagtggtcaataatggtcaataacccacacctataggctaagctataccatcacctatagcataaggaagcgggggtgtataggccccaagccaaaaacagtatagcatgcataagagccaaaggggtgtgcctatagactctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacatggcggtaatgttggacatgagccaatataaatgtacatattatgatatggatacaacgtatgcaatggccactgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaatt

SEQ ID NO: 4 - DNA, 7334 bp, Artificial sequence
  Multicistronic plasmid vector
attattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcatctatgatttctctcattacttcctcacatgttggaggcattttctctccctctgcacttaatagcctatcttgctttaatttcttcccactcttggaggctaggtttggtttggtgggctgatgagggagggagagaccgctccaagtactttagcgggtgggattgaaggcggagccctcctgagctatgagtgtcctatgagtggggctggaactaagaaccaggggcgtggacagggtgtgtcacagagaaggggatgtgcctgcttctttctggcccaggaggaaccgggtcaattcttcagcacctgggtaccacacaaaaaaccaacacacagatctctagagctctgatcttttattagccagaagtagatctggatccagcctgaatggcgcggccgcttgcacggtctaagagacctcccggggcactcgcaagcaccctatcaggcagtaccacaaggcctttcgcaacccaacgctactcggctagcagtcttgcgggggcacgcccaaatgaccggacatagagtgggtttatccaagaaaggacccagtctttccggtaattccggtgtactcaccggttccgcagaccactatggctctcccgggaggggggggcctggaggcttcgaagatatcatccgtcgaggaattctttgccaaaatgatgagacagcacaacaaccagcacgttgcccaggagctgtaggaaagagaagaaggcatgaacatggttagcagaggggcccggtttggactcagagtattttatcctcatctcaaacagtgtatatcattgtaaccataaagagaaaggcaggatgatgaccagggtgtagttgtttctaccaataagaatatttccacgccagccagaatttatatgcagaaatattctaccttatcatttaattataacaattgttctctaaaactgtgctgaagtacaatataatataccctgattgccttgaaaaaaaagtgattagagaaagtacttacaatctgacaaataaacaaaagtgaatttaaaaattcgttacaaatgcaagctaaagtttaacgaaaaagttacagaaaatgaaaagaaaataagaggagacaatggttgtcaacagagtagaaagtgaaagaaacaaaattatcatgagggtccatggtgatacaagggacatcttcccattctaaacaacaccctgaaaactttgccccctccatataacatgaattttacaatagcgaaaaagaaagaacaatcaagggtccccaaactcaccctgaagttctcaggatcggtcgatcgagctagctacccagcttggaggtgcacaccaatgtggtgaatggtcaaatggcgtttattgtatcgagctaggcacttaaatacaattatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacatttccccctccctatgcaaaagcgaaactactatatcctgaggggactcctaaccgcgtacaaccgaagccccgcttttcgcctaaacacaccctagtcccctcagatacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcagcagagcagattgtactgagagtgcaccatacaagctcaggatctgcgatgataagctgtcaacatagcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattatccgctagctcccggccgccatggcggccgcgggaattcgattgatcgatcgagagaaatgttctggcacctgcacttgcactggggacagcctattttgctagtttgttttgtttcgttttgttttgatggagagcgtatgttgctagcaccggtcatggctgcgccccgacacccgccaacacccggtgacgcgccctgacgggcttgtctgctcccggcatccgcttacagacaagctgtgaccgtctccgggagctgcatgtgtcagaggttttcaccgtcatcaccgaaacgcgcgaggcagccggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgcgtacggaaattcgagctcggtacccggggatcctttaaatgtggcaagcttatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcggaattggccgccctagatgctcgaggatcctctagagtcgacctgcagaaaagacccatggaaaggaacagtctgttagtctgtcagctattatgtctggtggcgcgcgcggcagcaacgagtactgctcagactacactgccctccaccgttaacagcaccgcaacgggagttacctctgactcttatcagaatacaacaactcagctgcctgcatcttcttctgccgctgccttaagtcttccatctgcgtcagccgtgcgagcccaatctccacgctcattttcagacacataccctaccgccacggccttgtgcggcacactggtggtggtgggcattgtgctgtgcctaagtctggcctccactgttaggagcaaggagctgccgagcgaccatgagccgctggaggcatgggaccagggctcggatgtggaagctccgccgctaccggagaagagcccatgtccggaacacgtacccgagattcgcgtggagatcccacgctatgtttaataaaaactgcgggcacgggggacggcgttgttgtatatgtgaatttgtaaataataaatgggaccccatcctgtaaaaatacagagtccgtgtcagtctctgaaggacagagtattggcatatagccaatagagatagttgtggcaaagagccatgttatggattagtaatggaaagtatcgtcaccaataggggagtggtcaataatggtcaataacccacacctataggctaagctataccatcacctatagcataaggaagcgggggtgtataggccccaagccaaaaacagtatagcatgcataagagccaaaggggtgtgcctatagactctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacatggcggtaatgttggacatgagccaatataaatgtacatattatgatatggatacaacgtatgcaatggccactgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaatt

(19) United States
(12) Patent Application Publication
(10) Pub. No.: US 2006/0263882 A1
(43) Pub. Date: Nov. 23, 2006

(54) Multi-cistronic vectors for gene transfer protocols

(75) Inventors: Vito Fazio, Roma (IT); Monica Rinaldi, Roma (IT); Laura Sonzogni, Zogno-Bergamo (IT); Giancarlo Tonon, Milano (IT); Gaetano Orsini, Gallarate-Varese (IT)

Correspondence Address: DARBY & DARBY P.C., P. O. BOX 5257, NEW YORK, NY, 10150-5257, US

(73) Assignee: Keryos SPA, Milano (IT)

(21) Appl. No.: 10/490,530

(22) PCT Filed: Oct. 10, 2002
(86) PCT No.: PCT/IT02/00646

§ 371 Date: Mar. 19, 2004

(30) Foreign Application Priority Data
Oct. 12, 2001 (IT) M12001A002110

(51) Int. Cl. C12N 15/86; C12N 15/09; C12N 5/06; A61K 48/00

(52) U.S. Cl. 435/455; 435/456; 435/325; 424/93.2

(57) ABSTRACT

The subject of the present invention is the construction of multicistronic eukaryotic plasmid expression vectors in which it is possible to express from two to four genes simultaneously and which are characterized by differently regulated bicistronic transcription units. The distinctive characteristic of these vectors is the presence of a CAP-independent translation initiation mechanism which is based on the ability of an IRES (internal ribosomal entry site) sequence to translate two proteins under the control of a single promoter. This family of multicistronic vectors can advantageously be used in various biotechnological applications in whcih the simultaneous expression of two or more genes is necessary, such as gene transfer protocols, DNA-immunization, or for the expression of different molecules in the same cell.

The subject of the present invention is the construction of multicistronic eukaryotic plasmid expression vectors in which it is possible to express from two to four genes simultaneously and which are characterized by differently regulated bicistronic transcription units. The distinctive characteristic of these vectors is the presence of a CAP-independent translation initiation mechanism which is based on the ability of an IRES (internal ribosomal entry site) sequence to translate two proteins under the control of a single promoter. This family of multicistronic vectors can advantageously be used in various biotechnological applications in which the simultaneous expression of two or more genes is necessary, such as gene-transfer protocols, DNA immunization, or for the expression of different molecules in the same cell.

### STATE OF THE ART

Various different approaches exist in the literature for achieving the co-expression of more than one gene within the same viral, retroviral, or DNA plasmid vector.

Each strategy described in the literature has the objective of preventing the problems which are associated with the use of several promoters in one vector for the expression of several proteins, such as the possibility of rearrangements or deletions, competitive interference of different promoters, or suppression of promoters. The presence of several promoters within the same vector in fact often leads to interference with transcription or to the dissociated expression of the different genes, with the possibility of a failure to express the gene of interest.

An alternative approach described consists in the use of fusion proteins, that is, two genes are fused “in frame” so as to obtain a chimeric protein. However, this mechanism is characterized by numerous problems which render it inapplicable in many cases, since two fused proteins do not always retain the same activity; moreover, the cell locations of the two starting proteins may be different and may render the expression and the functionality of the individual proteins of interest problematical.

A second alternative is the construction of a polycistronic unit composed of many encoding sequences in close proximity to one another so as to favour the translation re-initiation mechanism. The limitation of this approach is that the efficiency of the translation of genes subsequent to the first gene is often drastically reduced.

The present invention describes a novel approach which is based on the use of viral IRES sequences which can solve all of the problems connected with the co-expression of several proteins within the same vector. These sequences permit independent initiation of the translation of each peptide by virtue of the mechanism of operation which consists in the translation of the first gene in CAP-dependent manner, whereas the second gene is translated in an IRES-dependent manner. This mechanism has been shown to lead to efficient transcription and translation of the second cistron without interference of any type with the expression mechanism of the first cistron.

In particular, the tetracistronic vector described in the present invention is characterized by the presence of two IRES sequences of different origin, this configuration being capable of increasing the stability of the vector and preventing competition between transcription and translation factors involved in the expression of the cloned proteins.

### DETAILED DESCRIPTION OF THE INVENTION

A novel family of multicistronic eukaryotic expression vectors, characterized by two distinct, complete, and differentially regulated bicistronic transcription units, has been discovered and is now described. These vectors are characterized by the presence of two different promoter/enhancer sequences, that is, cytomegalovirus (p/eCMV) and Rous sarcoma virus (pRSV), which guide the transcription of recombinant cDNAs independently. The distinctive characteristic of the bicistronic vectors according to the present invention is connected with the presence of an alternative, CAP-independent translation-initiation mechanism which is based on the ability of the IRES sequence (the internal ribosome entry site) to translate two proteins under the control of a single promoter. The development of tricistronic or tetracistronic vectors derived directly from the above-mentioned bicistronic vectors and having the common property of being able to express different proteins, with the possibility that one of the proteins encoded may modulate the effect of others, is also described.

The efficiency of expression of these vectors has been demonstrated in vitro in various gene-transfer protocols, by means of COS cell transfection, by analyzing the expression of reporter genes.

These multicistronic vectors can be used in all biotechnological applications in which it is necessary to express two or more genes simultaneously such as, for example, gene transfer, immunization by administration of plasmid DNA for both prophylactic and therapeutic purposes, and for the expression of different molecules which modulate the effects of one another.

The ability of the IRES elements to promote the internal initiation of RNA translation has facilitated the expression of two or more proteins by a polycistronic transcription unit in eukaryotic cells. Expression vectors used to co-express genes often lead to transcriptional interference and/or to dissociated gene expression, with a fraction of the cells selected expressing only one of the genes of interest. The co-expression of two proteins, in which one is a reporter gene, a selectable marker, an antigen, or any molecule with immunomodulating or immunostimulating activity, is often a requisite of biotechnological applications; in particular, many applications in gene therapy require the coordinated release of more than one protein.

In recent years, to prevent the problems associated with the stability of different mRNA transcripts, bicistronic vectors containing IRES elements have been developed and have been applied to a variety of experimental situations from cell cultures to transgenic animals. The subject of the present invention is the construction of a novel series of plasmids for gene therapy and for the expression of proteins, characterized in that they contain regulator elements of viral origin or from eukaryotic genes. These sequences are contained in a compact and relatively small plasmid no larger than 8000 bp; the small size of the vector permits the introduction of genes of considerable size without compromising transfection efficiency, or the production of plasmids on a large scale from E. coli strains.

The plasmids of the present invention are characterized by one or two independent transcription units each of which consists of a strong viral promoter/enhancer (p/eCMV or pRSV), intron sequences (CMV-Intron A or rabbit β-globin Intron) to increase the stability of the transcripts thus obtained, a viral IRES, and an efficient transcription termination element (the polyadenylation site of SV-40 or the terminator of the growth hormone BGH, but it may also be the terminator derived from the rabbit β-globin gene mRGB). Both of the transcriptional units contain specific rare restriction sites in which a recombinant cDNA can easily be cloned.

In these novel vectors, transcriptional interference is prevented by the use of promoters and polyadenylation signals of different origins and by introducing pause sites at the end of the first translation cassette to terminate transcription effectively.

A distinctive characteristic of the vectors of the invention is that each transcription unit contains a different IRES sequence which is derived from the encephalomyocarditis virus (ECMV) or from the hepatitis C virus (HCV) and which can be used to assemble artificial eukaryotic operons in which the first cistron is translated in a conventional CAP-dependent mechanism and the subsequent cistron is based on CAP-independent translation initiation. It has in fact unexpectedly been found that the presence of different IRES sequences increases the stability of the vector and prevents competition for factors which act in trans on the sequence. These IRES elements are known to be amongst the strongest translation inducers and show little tissue tropism (Borman A. M. et al. “Comparison of picornaviral IRES-driven internal initiation of translation in cultured cells of different origins” Nucleic Acids Research. 25 (5): 925-932, 1997).

The vectors according to the present invention are constructed, on the basis of a prokaryotic vector pUC19, with a gene for kanamycin resistance and a replication origin (ori) based on pUC19 for amplification and maintenance in bacteria.

Finally, the possibility of cloning with 4 different and rare restriction sites enables any cDNA to be inserted in these plasmids and enables them to be used for many different biotechnological and therapeutic applications.

The main subject of the present invention is therefore represented by a multicistronic recombinant plasmid vector for the expression of at least two, identical or different, proteins of interest, containing at least one eukaryotic expression cassette comprising, in the reading stage, a promoter/enhancer sequence, an intron sequence, a cloning site, a viral IRES sequence, a cloning site, and a chain terminator.

Preferably, the IRES sequence comes from the encephalomyocarditis virus or from the hepatitis C virus, the promoter/enhancer sequence is p/eCMV or pRSV, the intron sequence is CMV-Intron A, or rabbit β-globin intron, and the terminators are polyA-SV40 and the BGH terminator (bovine growth hormone terminator, but it may also be the terminator derived from the rabbit β-globin gene mRGB).

According to one of the preferred aspects of the invention, the promoter/enhancer sequence and the intron sequence may be fused to one another; in particular, the promoter/enhancer sequence p/eCMV is preferably fused to the intron sequence CMV-Intron A.

If the vector contains two distinct translation cassettees, the two IRES sequences are preferably of different viral origin; normally, one cassette will contain the IRES sequence coming from the encephalomyocarditis virus and the other cassette will contain the IRES sequence coming from the hepatitis C virus. Similarly, one cassette will preferably contain the promoter/enhancer sequence p/eCMV and the other cassette the sequence pRSV, and one cassette will contain the intron sequence CMV-Intron A and the other rabbit β-globin intron. Similarly, one cassette will contain the poly A-SV40 terminator and the other the bovine growth hormone (BGH) terminator, but it may also be the terminator derived from the rabbit β-globin gene (mRGB). The preferred cloning sites are: SalI, XhoI, BsiWI, NotI, BstBI, EcoRVm, PacI and StuI.

In particular, the plasmid vectors having sequence: SEQ ID NO. 1 corresponding to PL 190, SEQ ID NO. 2 corresponding to PL 178, SEQ ID NO. 3 corresponding to PL 249, and SEQ ID NO. 4 corresponding to PL 250 constitute a preferred aspect of the present invention. Further subjects of the invention are represented by the eukaryotic host cells containing at least one plasmid vector as defined above, a method for the expression of at least two eukaryotic proteins, comprising the culture of the host cell (and, optionally, the recovery of the proteins), as well as the use of the plasmid vectors according to the present invention in gene transfer, in gene therapy, and in DNA immunization.

Further aspects of the invention will become clear from the following test section which should not be considered as limiting thereof.

### EXAMPLES

System of Selection with Prokaryotic Marker

The gene Amp^{R} was eliminated from the vector pUC19 (New England, Biolabs) by digestion with AlwNI and SspI and the remaining plasmid was ligated to the gene for kanamycin resistance, previously recovered from the vector pET39 (Novagen) by digestion with Asp700 and AlwNI, to give Pl-62 (2583 bp).

Purification of the Plasmid.

DNA contained in the transformed DH5-α E. coli strain (SUPE44,ΔlacU169(Φ80 lacZΔM15), hsdR17, rec A1, end A1, gyrA96, thi-1, relA1) was grown in LB medium, supplemented with 30 μg/ml of kanamycin, and the plasmid DNA was purified with the Clontech Miniprep plasmid kit. The DNA was quantified by UV spectrophotometry and its purity was checked by calculating the ratio of the absorbencies at 260 nm and at 280 nm.

Cloning Techniques

All of the cloning techniques were performed in accordance with the conventional methods described (Sambrook J., et al (1989) Molecular cloning: A laboratory Manual, 2nd ed., Cold Spring Harbor Laboratory Press, Cold Spring Harbor, N.Y.). Restriction and modification enzymes were obtained from New England Biolabs, Inc. and used as recommended by the vendor.

Cloning of the Reporter Genes

The DNA encoding for the reporter genes were amplified by PCR with the use of primers containing specific restriction sites to favour the integration of the genes in the cloning sites of the two vectors. The gene encoding for EGFP-1 (735 bp) was amplified from the vector pEGFP (Clontech) with the primers EGFP-1 (ACGCGTCGACgccaccattggtgagcaag; SalI site underlined, lower case=pairing) and EGFP-2 (CCTCCTCGAGttacttgtacagctcgtc; XhoI site underlined, lower case=pairing), digested with the SalI and XhoI restriction enzymes and ligated to the SalI and XhoI sites (MCS1) of Pl-163, to give the vector Pl-174 (5961 bp), and with the primers GFP-178-A (AGCTGATATcgccaccatggtgagcaa: EcoRV site underlined, lower case=pairing) and GFP-178-B (ACCGTTCGAAttacttgtacagctcgtc; BstBI site underlined, lower case=pairing), digested with the EcoRV and BstBI restriction sites and ligated to the EcoRV, BstBI (MCS3) sites of Pl-178, to give the plasmid Pl-189 (5200 bp). The gene encoding for luciferase (1630 bp) was amplified by PCR from the plasmid p 187 (Clontech) with the primers Luc-1 (GTATCGTACGtggtctagaattacacggcg; BsIWI site underlined, lower case=pairing) and Luc-2 (TACTCGTACGtttccatggaagacgccaa; BsIWI site underlined, lower case=pairing) and ligated to the plasmid Pl-174, previously digested with BsIWI (MCS2) to give the plasmid Pl-177 (7636 bp) and with the primers Lucif-1 (AATGCGGCCGCcatggaagacgccaaa; NotI site underlined, lower case=pairing) and Lucif-2 (ATAGCCATTCAGGCattacacggcgatctttc; BglI site underlined, lower case=pairing), digested with NotI and BglI and ligated to the NotI and BglI sites (MCS4) of Pl-189 to give the plasmid vector Pl-193 (5200 bp). All of the plasmids obtained were checked by restriction and sequence analysis.

Transfection Test

To demonstrate the activity of the transcriptional units inserted in the vectors pl-177 and pl-193 and containing the cDNA for luciferase and EGFP-1 as described above, and consequently to confirm the correct activity of the IRES disposed between the two cDNAs, the vectors obtained were transfected into a eukaryotic cell line (COS) of simian renal cells.

The COS cells were grown in single-layer conditions on non-selective DMEM medium in the presence of 10% bovine foetal serum and were kept at 37° C. in a humidified environment conditioned with 10% of CO₂. The COS cells, which were kept in culture at a concentration of between 1×10⁵ and 3×10⁶, were transfected by the DNA precipitation by means of calcium phosphate [(Ca₃ (PO₄)₂] method, with the use both of the recombinant plasmid pl-177 and of the control vector pl-163 at a final concentration of 10-20 μg/ml. The cells used for the transfection were seeded 24 hours previously at a density of 0.5-1×10⁶ per 10 cm plate. The culture medium was renewed 4-8 hours before and 8-10 hours after transfection.

The same method was followed with the vector pl-193.

Discussion

In the present invention, the strategy for the construction of a novel family of multicistronic vectors usable for various biotechnological and therapeutic applications is described. The family of vectors was obtained by the construction of two basic bicistronic vectors containing constitutive promoters of viral origin and different transcriptional elements, correlated with high levels of expression and with an increased stability of the yield of the transcripts. All of the plasmids constructed are based on the skeleton of the vector pUC19 which contains the replication origin ColE1 for the production of a large number of plasmid copies per bacterial cell, and which was modified by replacing the endogenous gene for ampicillin resistance which is used for an appropriate selection in bacterial systems (e.g. E. coli) with the gene for kanamycin resistance.

The vectors commonly described in the literature, which are based on a selection system that uses ampicillin resistance, would not in fact be acceptable by the International regulatory authorities (FDA and WHO) for use in human gene therapy since ampicillin could confer resistance to beta-lactamic antibiotics as well as inducing anaphylactic shock in sensitive individuals if it were present in traces as a contaminant of the plasmid DNA.

The vectors of the present invention are therefore characterized by the presence of the gene which encodes for kanamycin resistance and by the absence of other sequences of bacterial origin, which have been eliminated to prevent possible adverse effects on the levels of expression of the constructs.

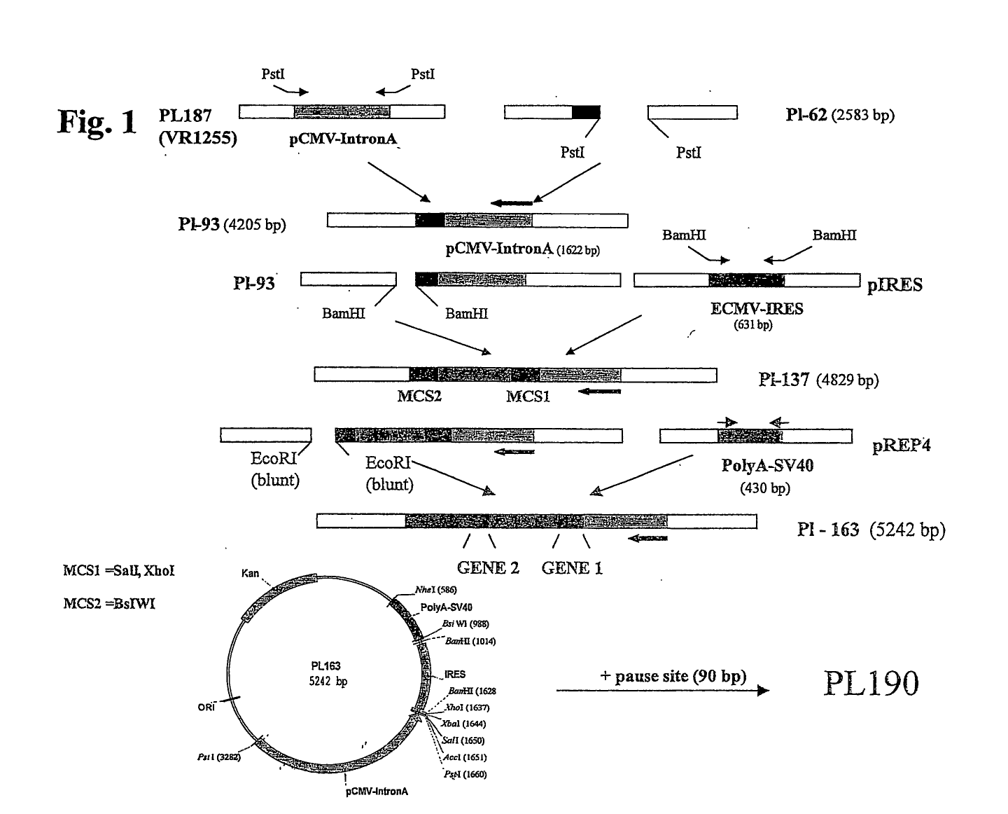

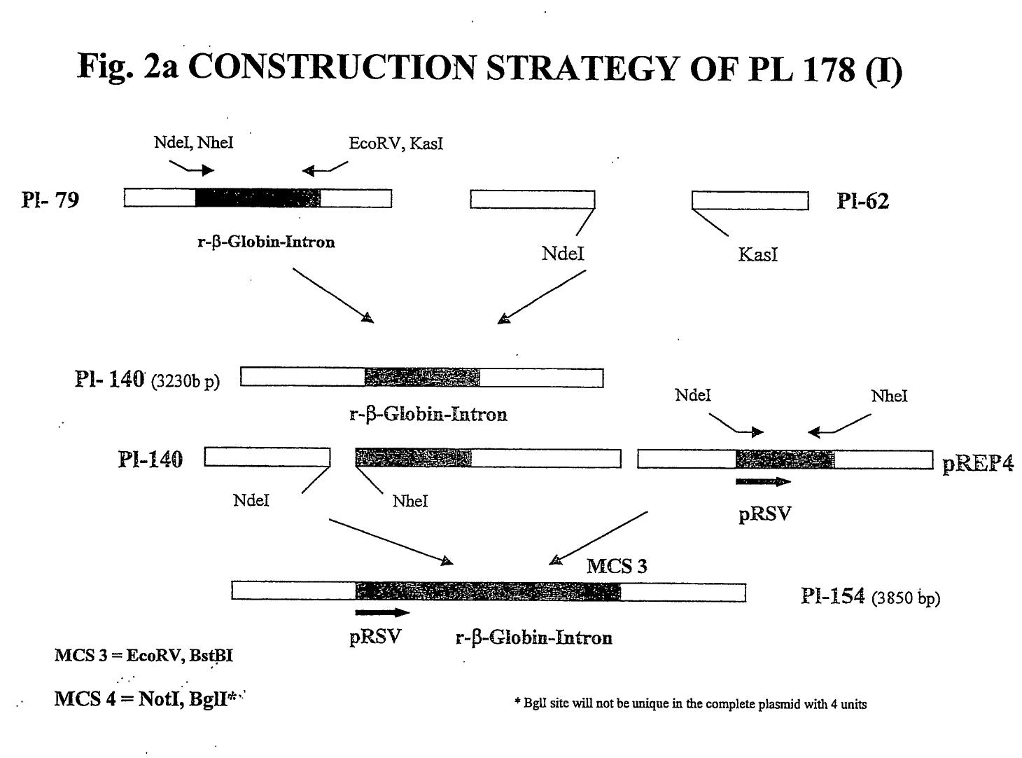

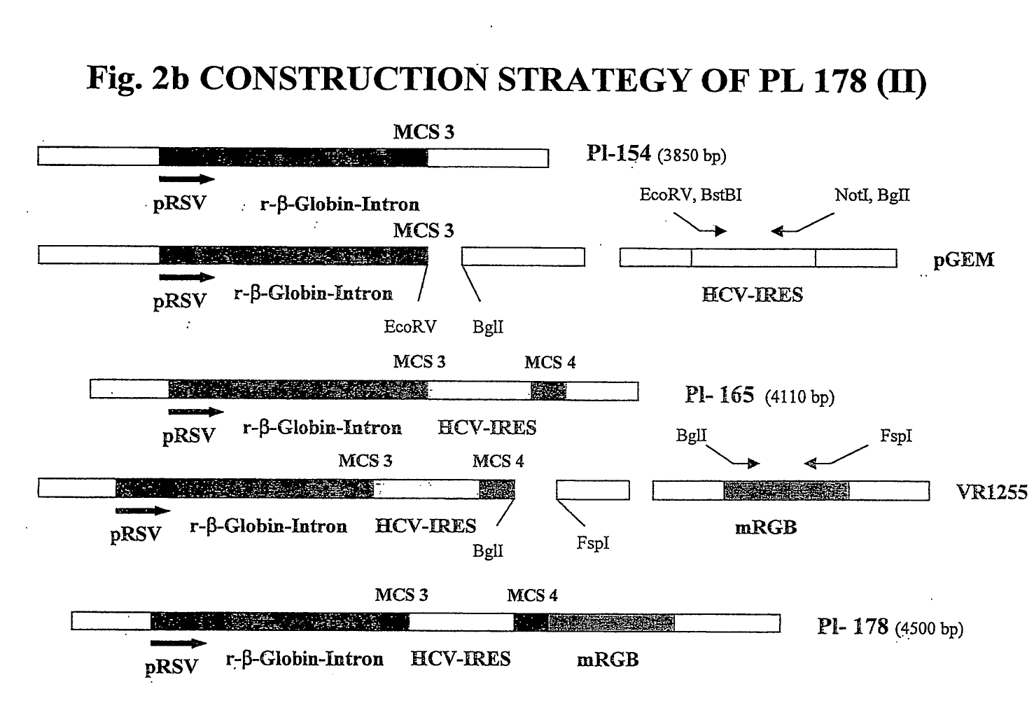

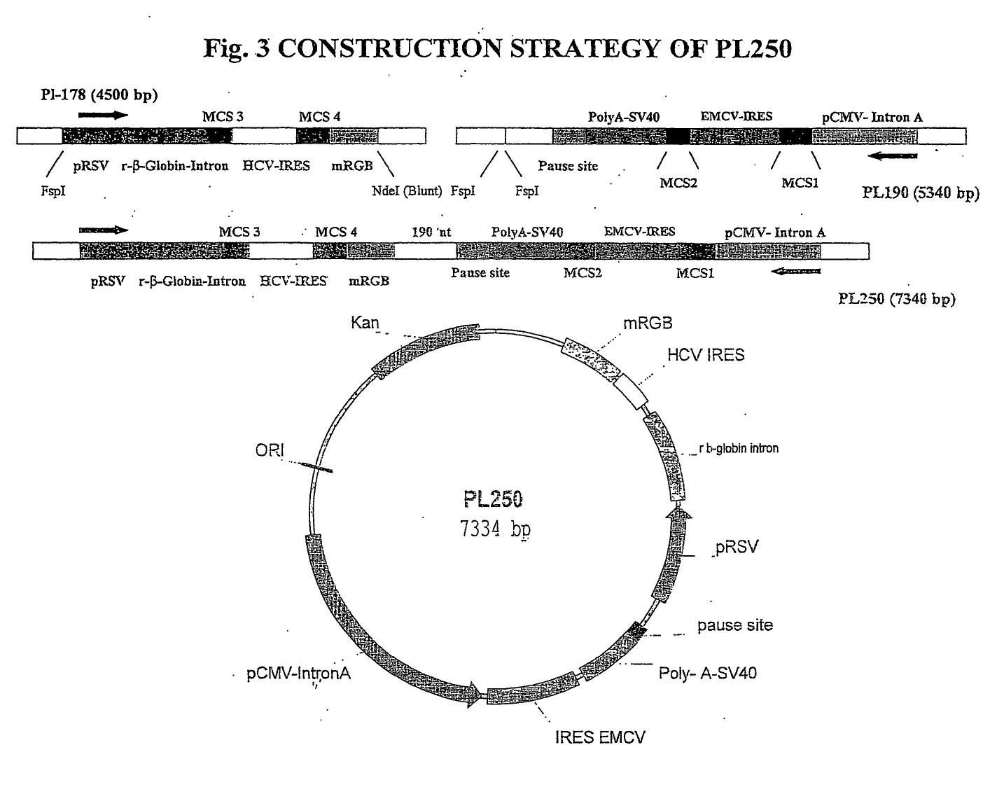

To construct the first bicistronic plasmid Pl-190, the various transcriptional elements, obtained by amplification by PCR, were ligated in the cloning sites present in the sequence of the basic vector pUC19 (FIG. 1), whereas all of the transcriptional elements of the bicistronic vector Pl-178 were cloned in the restriction site FspI of pUC19 which is located about 200 nucleotides downstream of the cloning region (poly-linker) of the vector pUC19 (FIG. 2). This was done for two reasons: firstly in order to be able to clone the complete transcription cassette of Pl-178 directly into the plasmid Pl-190 to give a tetracistronic vector called Pl-250 (FIG. 3), and secondly to create a distance of about 200 nucleotides between the two units in order to be sure that the RNA polymerase II is stopped after the termination signal present in the first transcription unit.

The tricistronic vector Pl-249 was obtained by eliminating the HCV-IRES sequence from the vector Pl-250 by digestion with restriction enzymes and adding the PacI and StuI restriction sites.

A pause site was also cloned downstream of the first transcription unit to eliminate the possibility of interference between the two transcription units. It has in fact been shown that, when a pause site is positioned immediately downstream of a strong polyadenylation signal, significant levels of transcription termination are achieved. (Levitt N., et al. “A pause site for RNA polymerase II is associated with termination of transcription”, EMBO. J. 10, 7, 1833-1842). The requirement for a functional pause site in the termination of the transcription by RNA polymerase II has been shown in several genes: human α2 globin, late adenovirus major, and late polyoma virus (Adami G., et al, EMBO J. 1998, 7, 2107-2116 “The slowing down or pausing of the elongation RNA polymerase beyond the 3′ processing signals of the gene is a key component of the termination process”; Eggermont J., Proudfoot N. J., “Poly(A) signals and transcriptional pause sites combine to prevent interference between RNA polymerase II promoters”. 1993 EMBO J. 12 (6): 2539-2548).

In the constructs described, the pause site of the human α2 globin gene was used; this is reported to be a region of 92 bp, very rich in A and characterized by an almost perfect repeated sequence (CA₄)₆ which acts in an orientation-dependent manner to decrease the speed at which the strong downstream processing signal is reached by the RNA polymerase. The 3′ half of the pause site does not have clear binding sites for proteins and neither can it be folded into a significantly stable RNA structure. (Moreira A. et al., “Upstream sequence elements enhance poly(A) site efficiency of the C2 complement gene and are phylogenetically conserved”. EMBO. J. 1995. 14 (15): 3809-3819).

When the region of 92 bp is disposed at the 3′ of an efficient polyadenylation site, it is possible to bring about not only a pause in transcription, but also significant levels of termination after the pause site. This region seems to interact with the polyandenilation site to facilitate transcription termination.

In order to construct the two transcription units, various transcriptional elements such as strong viral promoters (p/eCMV and pRSV), intron sequences (CMV-Intron A and r-β-globin intron), polyadenylation sites, and IRES elements were used. The promoters and the termination signals used were of different origins in order to eliminate any possibility of transcriptional interference.

Most of the commercial vectors for expression in mammals carry a promoter which is derived from pathogenic viruses. Although these elements are derived from viruses, they have become very useful elements for use in gene therapy protocols and for genetic immunization, by virtue of their good capability to initiate transcription in many mammalian tissues. The promoter/enhancer of cytomegalovirus (p/eCMV) and the promoter of Rous Sarcoma virus (pRSV) are two very strong viral promoters and are the promoters which are most commonly used since they induce a strong constitutive expression in various cell types. The two promoters show a similar strength, permitting the co-expression of two or more genes and preventing difference in expression when used together in the same vector.

With reference to the beneficial effects achieved by the use of intron sequences for the expression of proteins, it has been shown that they increase the expression of heterologous genes in vitro. (Chapman et al. “Effect of intron A from human cytomegalovirus (Towne) immediate-early gene on heterologous expression in mammalian cells”. 1991.NAR 19 (14): 3979-3986). Their effect on transcription expression has been associated principally with an increased rate of polyadenylation of the RNA, and with the nuclear transport associated with the splicing of the RNA. Moreover, the intron sequences have an important effect on antigen expression and have been shown to increase gene expression in various transfection systems. The positive effect of intron A and of rabbit β-globin intron on protein expression may be due to the regulation of upstream elements by promoter/enhancer activity, or to the contribution to the processing of stable mRNAs. (Huang M. T. and Gorman C. M. “Intervening sequences increase efficiency of RNA 3′ processing and accumulation of cytoplasmic RNA”. 1990. NAR. 18: 937-946). Another important element which is necessary for correct and efficient transcription termination is constituted by the polyadenylation sites. Polyadenylation is an essential process for the production of stable mRNAs and for increasing mRNA translation efficiency which may be variable according to the various terminators which are used. Moreover, when the transcription initiation rate is increased with the use of strong promoter/enhancer elements, the transcription termination process may become a limiting factor for transcription efficiency; (Jackson R. J. and Standart. “Do the poly(A) tail and 3′ untranslated region control mRNA translation?”. Cell. 1990; 62: 15-24). The late polyadenylation signal of SV40 is very efficient and increases the stability and the levels of RNA about 5 times more than the initial poly-A SV40 signal. (Carswell S. and Alwine J. C. “Efficiency of utilization of the simian virus 40 late polyadenylation site: effects of upstream sequences”. 1989. Molecular and Cellular Biology. 9: 4248-4258). In the plasmids described, this polyadenylation signal was located just downstream of the cloning site so as to facilitate efficient processing of the cloned genes which might not have an efficient polyadenylation signal.

The other polyadenylation site used in the vectors of the invention is the terminator derived from bovine growth hormone (BGH), which contains the essential polyadenylation signal and downstream termination elements, but it may also be the terminator derived from the rabbit β-globin gene (mRGB). (Levitt et al., “Definition of an efficient synthetic poly(A) site”. 1989, Genes and Dev. 3: 1019-1025). The presence of these optimized transcription terminators has been shown to increase protein expression, compared with other terminators (Hartikka J. et al. “An improved plasmid DNA expression vector for direct injection into skeletal muscle” Human Gene Therapy 1996/7: 1205-1217). The distinctive characteristic of the family of plasmid vectors constructed in accordance with the invention is the presence of two different IRES virus sequences, EMCV-IRES and HCV-IRES, which are used to bring about translation of the second cistron in the bicistronic mRNAs.

The internal ribosome entry sites (IRES) are elements which act in cis employing the small ribosomal subunit in an internal start codon present in the mRNA with the aid of cell factors which act in trans. This region can function as the initiator of an efficient translation of the reading code with no effect on the CAP-dependent translation mechanism which regulates the translation of the first cistron. The mechanism of the two processes also clearly differs from the use of different cell factors.

The IRES elements may adopt a particular secondary structure which catalyzes the assembly of the ribosomes and their scanning. Some mammalian genes contain IRES elements to ensure translation at specific stages of normal development or when translation is suppressed as a result of an infection. (Birnstiel M. L. et al., “Transcription termination and 3′ processing: the end is in site.” 1985 Cell. 4: 349-359). Pathogenic viruses often redirect cell translation resources of the host towards viral replication, destroying the CAP-dependent translation mechanism of the host and translating their transcripts in an IRES-mediated manner. Although most of these viral messengers are monocistronic, their IRES elements can be used to assemble artificial eukaryotic operons in which the first cistron is translated in a conventional CAP-dependent mechanism and the subsequent cistron in accordance with a CAP-independent translation initiation mechanism. (Fussenegger M. et al. “regulated multicistronic expression technology for mammalian metabolic engineering”. Cytotechnology 1998. 28: 111-125).

The ability of the IRES elements to promote internal initiation of RNA translation has facilitated the expression of two or more proteins by a polycistronic transcription unit in eukaryotic cells (Borman A. M. et al. “Comparison of picornaviral IRES-driven internal initiation of translation in cultured cells of different origins” NAR. 1997. 25 (5): 925-932); in most cases, the efficiency of the expression of the second gene is increased (Gallardo H. F. et al. “The internal ribosome entry site of the encephalomyelitis virus enables reliable co-expression of two transgenes in human primary T lymphocytes”. Gene Therapy. 1997. 4:1115-1119).

In the encephalomyocarditis virus (EMCV), the IRES region, which has been included in accordance with a general classification in Picornavirus type II IRES, consists of about 450-600 nucleotides of the non-translated region at 5′, and terminates at 3′ with an element of 25 nucleotides which consist of a conserved UUUC motif, followed by a more variable portion which is rich in pyrimidine and a spacer which is poor in G and, finally, an AUG triplet which is considered to be the actual ribosome entry site. The presence of this region facilitates the creation of a bicistronic vector since it permits the translation of two ORFs by a single messenger. (Jang S. K. et al., “A segment of the 5′ non-translated region of encephalomyocarditis virus RNA directs internal entry of ribosomes during in vitro translation”. J. of Virology 1998. 62 (8): 2636-2643). The IRES sequence of the hepatitis C virus (HCV) behaves like a type II IRES such as the IRES of ENCV and of FMDV which have been described as the best candidates for guiding the initiation of internal translation. This region is known to be able to initiate translation of the polyprotein of HCV by means of an internal ribosome entry site (IRES) which involves most of the non-translated regions. The IRES sequence of HCV is greatly dependent both on the primary sequence of this segment and on its ability to form complexes of secondary and tertiary structure of the RNA; in this region, the majority of HCV viral genotypes possess 5 AUG codons which are not used for initiating translation.

Various works show that the IRES of HCV guides translation initiation in all of the cell lines tested, including those which are not derived from primates. Moreover, the IRES of HCV, as shown in other type II IRES, is much more efficient in vivo than in vitro. The minimum HCV-IRES sequence is still debatable. “Fukushi S. et al. “Complete 5′ non coding region is necessary for the efficient internal initiation of hepatitis C virus RNA” BBRC. 1994. 199 (2): 425-432; Tsukiyama-Kohara K. et al “Internal ribosome entry site within hepatitis C virus RNA”. J. of Virology. 1992. 66 (3): 1476-1483). Various studies describe the IRES sequence extending approximately from nucleotide 44 to 354 of the HCV genome. The inventors elected to use the nucleotide sequence included from nucleotide 109 to 341 (230 bp of the viral genotype 1b) since it has been shown to be the minimum functional region. (Collier A. J. et al. “Translation efficiencies of the 5′ untranslated region from representatives of the six major genotypes of hepatitis C virus using a novel bicistronic reporter assay system”. 1998. Journal of General Virology. 79: 2359-2366).

The use of a short IRES segment is particularly important for the construction of bicistronic vectors since it increases the dimensions of the space for the insertion of a therapeutic gene. The functionality of the vectors of the invention was tested in vitro by transfecting COS cells, with the use of the CaCl₂ method, and cloning the EGFP and luciferase reporter genes which are easily detectable both in vitro and in vivo.

### CONCLUSIONS

The novel vector family described in the present invention represents a versatile tool which enables novel gene combinations to be created in a single vector. The ability to co-express two or more genes in a single vector is significantly more effective and advantageous than the use of two separate vectors. This family of polycistronic vectors may have many different applications in fields which include approaches to gene therapy, co-expression studies, analysis of the synergic effect of two genes in a particular cell system, and co-expression of two or more subunits of a protein.

The use of a single vector simplifies and dramatically augments co-transfection studies since it enables time to be saved in the process for the production of stable transfectants in a single selection step in order to produce co-transfectants for expressing both of the genes and to achieve similar levels of expression for two proteins.

The novel vectors described in the present invention are therefore usable to express two or more genes in various biotechnological applications, in particular for gene therapy applications, such as cancer gene therapy, immunogenic therapy, and DNA vaccination. In all of these applications the multigenic approach has in fact shown greater efficiency than the monogenic approach.

FIG. 1 Schematic Illustration of the Construction of the vector Pl-190

a) p/eCMV-intron A (1622 bp) was amplified by PCR from the vector Pl-187 with the primers EP1A-1(AAAACTGCAGtgggccattgcatacgttgta; PstI site underlined, lower case=pairing) and EP1A-2 (CAAACTGCAGaaaagacccatggggggaaag; PstI site underlined, lower case=pairing), digested with PstI restriction enzyme and ligated to the PstI site of the cloning site of Pl-62 to give the plasmid Pl-93 (4205 bp).

The promoter p/eCMV was cloned in the same direction as the promoter LacZ of the vector pUC19; the same was done for all of the other cloned fragments downstream of the p/eCMV promoter.

b) ECMV-IRES (631 bp) was amplified from pIRES (Invitrogen) with the primers pIRES-1 (AGCGGATCCtcgagcattctagggcggaatt; BamHI site underlined, lower case=pairing) and pIRES-2 (GCGGATCCtttaaatgtggcaagcttattcatcgt; BamHI site underlined, lower case=pairing), digested with BamHI restriction enzyme and ligated at the BamHI site of Pl-93 to give the vector Pl-137 (4829 bp).

c) The polyA-SV40 fragment (430 bp) was amplified by PCR from the vector pREP4 (Invitrogen) with the primers poly-2A (TCCGTACGcagacatgataagatacattg; BsIWI site underlined, lower case=pairing) and poly-3A (ATCCGCTAGCaccggtcatggctgcgc; NheI site underlined, lower case=pairing) and ligated to the Pl-137 vector previously digested with EcoRI and treated with Klenow modification enzyme. This cloning resulted in the vector Pl-163 (5242 bp). The BsIWI and NheI restriction sites were introduced into the primers to facilitate the following cloning steps.

d) The pause site fragment (98 bp) was amplified from the vector P-GEM-P (obtained synthetically from Baseclear, B H Leiden, The Netherlands) with the primers PLS-1 (CTAGCTAGCaacatacgctctcc; NheI site underlined, lower case=pairing) and PLS-2 (TCAGCTAGCagagaaatgttctggcac; NheI site underlined, lower case=pairing) and ligated to the vector Pl-163, previously digested with NheI. The cloning gave rise to the plasmid Pl-190 (5340 bp).

All of the vectors obtained were checked by restriction and sequence analysis. MCS=multiple cloning sites. MCS1=SalI, XhoI; MCS2=BsIWI.

FIGS. 2a/b Schematic Illustration of the Construction of the Vector Pl-178

a) For the construction of the plasmid Pl-178, all of the fragments were amplified by PCR so as to create specific restriction sites for integrating the fragments in the vector Pl-62 and also to create new unique cloning sites for the subsequent cloning steps. Rabbit β-globin intron (700 bp) was amplified from the vector Pl-79 with the primers Intron-1 (ATGGCGGCCGATATCatccgtcgaggaattcttt; KasI site underlined, EcoRV site underlined, lower case=pairing) and Intron-2 (ATTCCATATGCTAGCtcgatcgaccgatcctgaga; NdeI site underlined, NheI site underlined, lower case=pairing) and ligated to the vector pl-62, previously digested with NdeI and KasI restriction enzymes. The cloning gave rise to the vector Pl-140 (3230 bp). The EcoRV and NheI restriction sites were introduced to facilitate the subsequent cloning steps.

b) pRSV (623 bp) was amplified from the vector pREP-4 (Invitrogen) with the primers pRSV-1 (CATGCTAGCtacccagcttggaggtgca; NheI site underlined, lower case=pairing) and pRSV-2 (ATTCATATGttgacagcttatcatcgcag; NdeI site underlined, lower case=pairing), digested with NdeI and NheI and ligated to the NdeI and NheI sites of Pl-140 to create the vector Pl-154 (3859 bp).

c) For the construction of the plasmid Pl-165, the fragment encoding the HCV-IRES region (260 bp) (Produced synthetically by Baseclear, B. H. Leiden, The Netherlands) was cut, by restriction with the enzymes BglI and EcoRV, from the vector pGEM+HCV-IRES and ligated to the BglI and EcoRV sites of the plasmid Pl-154 to give the vector Pl-165 (4110 bp).

d) The BGH fragment (390 bp) was amplified from the vector Pl-187 with the primers mRGB-1 (ATAGCCATTCAGGCtggatccagatctacttc; BglI site underlined, lower case=pairing) and mRGB-2 (GACTTGCGCAtcctatgaatttctctccattac; FspI site underlined, lower case=pairing), digested with BglI and FspI and ligated to the BglI and FspI sites of Pl-165 to give the vector Pl-178 (4500 bp).

All of the vectors obtained were checked by restriction and sequence analysis. MCS=multiple cloning sites. MCS3=EcoRV, BstBI; MCS4=NotI, BglI (The BglI restriction site will not be a unique site in the tetracistronic vector.)

FIG. 3 Construction of the Multicistronic Vectors

For the construction of the plasmid Pl-150 which can potentially express 4 genes, the vector Pl-178 was digested with FspI and NdeI, treated with Klenow, and cloned in the FspI site of Pl-190 to give the final plasmid Pl-250.

For the construction of the plasmid Pl-249, which can potentially express 3 genes, the HCV-IRES sequence was eliminated from the vector Pl-250 by restriction with the enzymes EcoRV-NotI and the PacI and StuI restriction sites were added.

1. Multicistronic recombinant plasmid vectors usable for the expression of at least two, identical or different, proteins of interest, containing at least one eukaryotic expression cassette comprising, in the reading stage, a promoter/enhancer sequence, an intron sequence, a cloning site, a viral IRES sequence, a cloning site, and a chain terminator.

2. A plasmid vector according to claim 1, including a transcription pause site disposed downstream of the chain terminator.

3. A plasmid vector according to claim 1, characterized in that the IRES sequence comes from the encephalomyocarditis virus or from the hepatitis C virus.

4. A plasmid vector according to claim 1, characterized in that the promoter/enhancer sequence is p/eCMV or pRSV.

5. A plasmid vector according to claim 1, characterized in that the intron sequence is CMV-Intron A or rabbit β-globin intron.

6. A plasmid vector according to claim 1, characterized in that the promoter/enhancer sequence and the intron sequence are fused to one another.

7. A plasmid vector according to claim 1, characterized in that the chain terminator is selected from polyA-SV40, BGH, and mRGB.

8. A plasmid vector according to claim 2, characterized in that the pause site is the human α2 globin gene.

9. A plasmid vector according to claims 1-6, characterized in that there are two expression cassettees.

10. A plasmid vector according to claim 7, characterized in that the two IRES sequences are of different viral origin.

11. A plasmid vector according to claim 8, characterized in that one cassette contains two IRES sequences coming from different viral genomes.

12. A plasmid vector according to claim 9, characterized in that one cassette contains the IRES sequence coming from the encephalomyocarditis virus and the other cassette contains the IRES sequence coming from the hepatitis C virus.

13. A plasmid vector according to claim 7, characterized in that one cassette contains the promoter/enhancer sequence p/eCMV and the other cassette contains the promoter/enhancer sequence pRSV.

14. A plasmid vector according to claim 7, characterized in that one cassette contains the intron sequence CMV-intron A and the other cassette contains rabbit β-globin intron.

15. A plasmid vector according to claims 7-12, characterized in that one cassette contains the promoter/enhancer sequence pCMV fused to the intron sequence hCMV-intron A, and the IRES sequence coming from the encephalomyocarditis virus and the other cassette contains the promoter/enhancer sequence pRSV, rabbit P-globin intron, and the IRES sequence coming from the hepatitis C virus.

16. A plasmid vector according to claims 7-12, characterized in that one cassette contains the sequence of the terminator polyA-SV40 and the other cassette contains the terminator BGH or mRGB.

17. A plasmid vector according to claim 1, characterized in that the cloning sites are selected from: SalI, XhoI, BsiWI, NoTI, BstBI, ECoRV, PacI and StuI.

18. A plasmid vector according to claim 1 having SEQ ID NO. 1, SEQ ID NO. 2, SEQ ID NO. 3 or SEQ ID NO. 4.

19. A plasmid vector according to claim 1, characterized in that at least one of the proteins is a reporter gene, a selectable marker, an antigen, or any molecule with immunomodulating or immunostimulating activity.

20. A plasmid vector according to claim 1 containing, in distinct cloning sites, the gene sequences encoding for the at least two proteins of interest.

21. Eukaryotic host cells, characterized in that they contain at least one plasmid vector according to claim 18.

22. A method for the expression of at least two eukaryotic proteins, comprising the culture of a host cell transformed in accordance with claim 19 and, optionally, the recovery of the proteins.

23. Use of a plasmid vector according to claims 1-18 in gene transfer, in gene therapy, and in DNA immunization.
